(19) United States
(12) Patent Application Publication
(10) Pub. No.: US 2004/0192706 A1
(43) Pub. Date: Sep. 30, 2004

(54) Method and compositions for treating anxiety

(76) Inventors: Janet Codd, Westmeath (IE); Bernard Beer, Cliffside Park, NJ (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/721,923

(22) Filed: Nov. 25, 2003

Related U.S. Application Data
(60) Provisional application No. 60/430,740, filed on Dec. 4, 2002.

(51) Int. Cl. A61K 31/519

(52) U.S. Cl. 514/259.3

(57) ABSTRACT

A method for treating anxiety in a patient by orally administering to said patient a daily dose of the active ingredient, 2-pyridinyl[7-(4-pyridinyl)pyrazolo[1,5-a]-pyrimidin-3-yl]-methanone or its pharmaceutically acceptable salts especially Ocinaplonin, in two separate divided administrations, with the first portion being administered in a slow release form and the remaining portion being administered in instant release form so as to maintain the blood levels of this active ingredient at therapeutically relevant levels to keep the anti anxiety effect in the patient during the periods between administrations of this active ingredient as well as pharmaceutical compositions containing this active ingredient for administration in connection with the above method.

### CROSS REFERENCE TO RELATED APPLICATIONS

This Application claims priority of U.S. Provisional Serial No. 60/430,740 filed Dec. 4, 2002, incorporated herein by reference.

### BACKGROUND OF INVENTION

Benzodiazepines remain widely used for the treatment of anxiety. Nonetheless, there are a number of side effects associated with benzodiazepines that limits their use in the treatment of anxiety disorders. For example, benzodiazepines produce muscle relaxation, sedation, and amnesia, actions that are generally considered undesirable in the management of anxiety disorder. An improved compound for treating anxiety disorders would relieve symptoms as effectively as benzodiazepines but lack these limiting and potentially dangerous side effects. One of these other types of compounds that can be used in this respect are the pyridinylpyrazolo[1,5-a]pyrimidinylmethanones which are anxiolytic agents such as disclosed in U.S. Pat. No. 4,521,422 and Vancouver et al., Experimental and Clinical Psychopharmocology 1994, Vol. 2, No. 3, pg. 225-233. However, these compounds are difficult to administer from the standpoint of patient compliance because, while achieving rapid peak blood levels, they are cleared from the bloodstream rather quickly, and would require dosing at least three times a day to achieve sustained, therapeutically relevant blood levels. Therefore, it is important that in addition, to achieving a rapid onset of action, blood levels of this pharmaceutical be maintained therapeutically until the activity resulting from the next administration of this pharmaceutical is achieved.

### SUMMARY OF INVENTION

In accordance with this invention a composition and a method of utilizing this composition has been developed for administrating the active ingredient 2-pyridinyl[7-(4-pyridinyl)pyrazolo[1,5-a]-pyrimidin-3-yl]-methanone or its pharmaceutically acceptable salts. This active ingredient in the form of its free base has the formula.

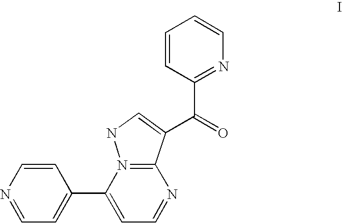

In accordance with this invention, a new method and composition for administrating the compound formula I (Ocinaplon) or its pharmaceutically acceptable salts to patients for anti-anxiety has been developed. When this compound is administered to a patient in accordance with this development, the blood levels are maintained at therapeutically relevant levels during the periods between the administration of this compound. In such a manner, there is no need to repeatedly administer the compound of formula I or its salt to maintain its anti-anxiety effect. In accordance with this invention, this anti-anxiety effect can be maintained throughout the course of a given day with only one or two daily administrations of this compound. Through the method and compositions of this invention, a new class of compounds, other than benzodiazepines, have been developed for treating anxiety.

### DETAILED DESCRIPTION OF THE INVENTION

In accordance with this invention a composition and method for administering the compound of formula I or its pharmaceutically acceptable salts to treat anxiety in patients in need of this treatment has been developed. This method involves orally administering to a human patient the compound of formula I or its pharmaceutically acceptable salt at a dose of from about 0.5 mg/kg to about 5.0 mg/kg of body weight. This is clearly achieved by utilizing a daily dosage regiment of from about 50 to 250 mg, preferably from about 8o mg to about 240 mg and most preferably from about 100 mg to about 240 mg In accordance with a preferred embodiment of this invention, the daily administration of the compound of formula I or its pharmaceutically acceptable salts may be carried out in from 1 to 3 separate administrations during a given day, preferably from one to two times per day. Each of these separate administrations contain a portion of the compound of formula I or its salt in a slow release form while the remaining portion of this active ingredient is administered in a rapid release form. During each of these separate administrations, the active ingredient in rapid release form is from about 1 to 4 times, preferably from about 2.5 to about 3.5 times, the weight of the portion in slow release form.

In accordance with this invention the compound of formula I or its pharmaceutically acceptable salts can be utilized to treat any form of anxiety including various anxiety disorders. Among the anxiety disorders which can be treated in accordance with the method of this invention include Generalized Anxiety Disorder and Panic Disorder listed in DSM-IV. The compound of formula 1 designates the active ingredient Ocinaplon. In accordance with this invention, the compound of formula I, when administered produces the aforementioned beneficial results of this invention.

For carrying out the method of this invention, a pharmaceutically oral unit dosage form has been developed which comprises the active ingredient separated into two compartments, the first of which contains the active ingredient together with a pharmaceutically acceptable carrier for rapid release and the second compartment contains the active ingredient and a carrier incorporated into a sustained release hydrophilic polymeric matrix which causes sustained release of the active ingredient from the second compartment. By the provision of such a method and unit dosage form, one is able to maintain an appropriate therapeutic blood level of the active ingredient in the blood stream of the patient. In the patient, this level is sustained throughout the day. In addition, there is no need for a patient to repeatedly administer throughout the day the compound of formula I or its pharmaceutically acceptable salt.

The compound of formula I may be administered as a free base or in the form of its pharmaceutically acceptable salt. Any conventional pharmaceutically acceptable acid addition salt such as the hydrochloric acid salt, the citric acid salt, etc. can be utilized. Other salts, which are preferred include, for example, hydrobromide, hydroiodide, sulfate, nitrate, phosphate and malate salts.

The compound of formula I or its pharmaceutically acceptable salt is administered in accordance with this invention in an oral unit dosage form. Any conventional oral unit dosage form can be utilized with the preferred oral unit dosage form being tablets. The daily dose for achieving the desired anti-anxiety effect by utilizing the compounds formula I is from about 50 to 250 mg, preferably from about 80 to about 240 mg, with about 100 mg to about 240 mg being especially preferred. In accordance with this invention, this total daily dose can be administered in from 1 to 3 separate administrations, preferably in from 1 to 2 separate administrations. Generally it is preferred that these separate daily dosages be approximately equal in the amount of active ingredient administered. As an example, if one wanted to administer a dosage of 50 mg of the active ingredient in two separate doses during the day, then each of the separate administrations can be about 25 mg On the other hand, where one wants to administer 240 mg total daily dose to a patient in two separate administrations during the day, then each separate administration should preferably be about 120 mg Also these two separate doses should preferably be administered approximately at equal time periods during the day (i.e. about 8 hours or 12 hours difference). However, the time period which separates these daily doses can be from about 6 to 14 hours, depending upon the strength of the dose, without any deleterious effect.

In carrying out this invention, it is important that the divided doses have a slow release portion and a rapid release portion and that weight of the rapid release portion be about, 1 to 4 times, preferably from about 2.5 to about 3.5 times, the weight of the slow release portion. Therefore the weight of the active ingredient administered for rapid release should be from about 1 to 4 times greater than the weight of the portion administered for slow release during each of these two divided dosages. This will allow stabilization and constant maintenance of the active ingredient in the blood system during the entire periods between administrations of the active ingredient. Generally it is preferred that the active ingredient administered for rapid release be about three times the weight of the portion administered for slow release.

In these separate administrations of this active ingredient, the amount of the active ingredient in slow release form and in the rapid release form can be respectively administered separately or in combination. The rapid release form for a given administration can be administered in one dosage form such as one capsule or one tablet or in separate tablets each totaling the amount to be administered through rapid release in this separate administration. For example, if one wishes to administer 90 mg of the active ingredient in rapid release form one can administer three tablets or capsules each containing a different amount or the same amount of this active ingredient, which amount total is 90 mg The same is true with respect to the administration of the sustained release form of the active ingredient. In any case during each of the separate administrations, the amount of active ingredient administered in rapid release form should be from about 1 to about 4 times the weight of the portion administered in sustained release form. During each of these two separate administrations, the active ingredient in rapid release form and sustained release form could be administered together or even separately, but within a short period of time.

In accordance with a preferred embodiment of this invention, oral unit dosage forms have been provided, such as tablets or capsules, for administering the active ingredient in both the sustained and rapid release forms as a single dosage unit. These oral unit dosage forms can be any conventional dosage unit preferably a tablet. Therefore during a single administration a patient can take one or two of these unit dosage forms containing both the active ingredient in sustained release and in rapid release forms. In accordance with this invention, oral dosage forms containing about 15 mg to about 250 mg of the compound of formula I or salts thereof can be prepared with the active ingredient in the rapid release portion being about from 1 to 4 times the amount of the slow release portions. The preferred oral unit dosage forms are those capsules or tablets having 120 mg of the active ingredient with 30 mg of the active ingredient in sustained release form and 90 mg of the active ingredient in rapid release form. Other preferred oral dosage forms include 40 mg capsules or tablets of the active ingredient, containing 10 mg in sustained release form and 30 mg in rapid release form. Another preferred form is 240 mg tablets or capsules of the active ingredient containing 80 mg of the active ingredient in sustained release form and 160 mg of the active ingredient in rapid release form. In this manner a simplified means of administration is provided for the combination of the slow and rapid release dosage forms to maintain the blood level of the compound of formula I constant throughout the period of treatment.

In preparing the oral unit dosage forms, preferably the tablets, containing the compound of formula I as an active ingredient, this active ingredient is mixed with a diluent, or carrier, usually present in about from 40 to 75 percent by weight of the immediate release composition. With slow release tablets the diluent is generally from 40 to 60 percent by weight. Among the preferred diluents, or carriers, is included lactose. In accordance with this invention any of the conventional diluents can be utilized. These oral dosage forms, particularly tablets, generally contain other conventional excipients such as binders, disintegrants, lubricants, or glidants, which are conventionally used in formulating such oral unit dosage form as tablets. The disintegrant may be one of several modified starches or modified cellulose polymers in particular, cross croscarmellose sodium as well as polyvinylpolypyrrolidone. The binder can be any conventional binder such as polyvinylpyrrolidone, microcrystalline cellulose.

In preparing the sustained release tablets, the active ingredient of formula I or its salt and the diluent or carrier are mixed together with one or more other additional formulation ingredients or excipients and further mixed with a hydrophilic polymeric sustained release matrix which causes the slow release of the active ingredient into the patient upon ingestion of the tablet. The hydrophilic slow release polymer that is used in accordance with this invention generally have has a viscosity in the range of about 100 cps to about 100,000 cps.

Any conventional polymeric hydrophilic sustained release matrix can be utilized. Among these are included hydroxypropylmethyl cellulose as well as other hydrophilic polymers such as hydroxypropyl cellulose, hydroxyethyl cellulose, carboxymethylcellulose, sodium carboxymethylcellulose, carboxyvinylpolymers, polyvinyl alcohols, glucans, scleroglucans, mannans, xanthans, carboxymethylcellulose and its derivatives, methylcellulose and, in general, cellulose, crosslinked polyvinyl pyrrolidone. The hydrophilic polymeric matrix which produces the slow release of the compound of formula I in accordance with this invention are generally incorporated into the slow release formulation in amount of from about 20 percent to about 35 percent by weight of the entire slow release formulation.

The tablets of this invention are prepared in accordance with the usual tabletting method except that the active ingredient and the carrier, or diluent, are ground to a particle size having a diameter of less than 250 microns. Any conventional means of grinding the formulation can be utilized to achieve this result. A preferred method of achieving this result is by passing the composition of the active ingredient, with or without the polymeric sustained release matrix and the diluent through a hammer mill with a sixty mesh screen at high speeds to produce this size reduction. With respect to the slow release formulation the mixture which is passed through the hammer mill includes the slow release matrix in the composition. After granulation, the composition can be compressed into tablets which either contain the active ingredient in slow release form or contain the active ingredient of rapid release form. This is accomplished by any conventional tabletting means such as compression utilizing a single layer tablet press. On the other hand, if it is desired to provide a bi-layer unit tablet, one can take both the slow release composition and the rapid release composition and tablet them into a single bi-layered tablet. In this method, one can utilize a bi-layer tablet press to formulate these two components into a single tablet. Any conventional means of forming a bi-layer tablet through compression of the active ingredient component mixture can be utilized to carry out this procedure.

The term “tablet” as used herein is intended to encompass compressed pharmaceutical dosage formulations of all shapes and sizes either coated or uncoated. Substances which may be used for coating include any of the classic coating material which can be titanium dioxide, talc, sweeteners and colorants.

This invention is further illustrated by the following examples. In the examples:

Polyplasdone is polyvinyl-polypyrrolidone.

Aerosil is colloidal silicon dioxide.

Kollidon 30 is poly-vinyl pyrrolidone (PVP).

Mag Stearate is magnesium stearate.

Methocel K100M is hydroxy propyl methylcellulose, 1,000,000 cps.

Methocel K100LV is hydroxy propyl methylcellulose, 1,000,000 cps.

### EXAMPLES

### Example 1

### Preparation of Rapid Release (RR) 60mg AND 90 mg Tablets

% BY WEIGHT BASED UPON
WEIGHT OF COMPOSITION

| Material | 60 mg RR Tablet | 90 mg RR Tablet |
|---|---|---|
| Ocinaplon | 18.6 | 27.1 |
| Fast Flow Lactose | 63.9 | 53.8 |
| Polyplasdone | 9.7 | 9.7 |
| Aerosil | 0.8 | 0.9 |
| Kollidon 30 | 3.1 | 4.5 |
| Mag Stearate | 1.0 | 1.0 |
| Opadry Brown | 2.9 | 2.9 |

Manufacturing Process for the Tablets

A. Weigh the Ocinaplon, Fast-flow lactose, half of the Polyplasdone, half of the Aerosil 200.

B. Blend in 10LV cone blender for ten minutes at 18 rpm.

C. Pass through a Fitzmill (hammers), 60 mesh screen; at high speed to produce particles having a diameter less than 250 microns.

D. Granulate in a fluid bed system (Glatt GPCG-3), using PVP (7.5% w/w aq soln) as the binder, to a target composition of 5% binder/95% milled blend.

E. Blend the granule in a V cone blender with the remainder of the Aerosil, and the remainder of the Polyplasdone for 5 minutes, before adding Magnesium Stearate, and blending for a further 3 minutes.

F. Manufacture the IR tablets using a Piccola tablet Press, using 9 mm round, normal concave tooling to a target hardness of 160 N (±30%), and the required target weight (±7%).

G. Coat the resultant tablets with a cosmetic coating of Opadry Brown (12% w/w aq soln), on a Vector Coater, to a target weight gain of 3%.

### Example 2

### Preparation of a 30 mg Sustained Release (SR) Tablet

| | % BY WEIGHT BASED UPON |
|---|---|
| | WEIGHT OF COMPOSITION |
| Material | 30 mg SR Coated Tablet |
| Ocinaplon | 19.4 |
| Methocel K100M | 11.6 |
| Methocel K100LV | 17.5 |
| Fast flo Lactose | 41.8 |
| Aerosil | 1.0 |
| Kollidon 30 | 4.8 |
| Mag Stearate | 1.0 |
| Opadry Brown | 2.9 |

Manufacturing Process for Ocinaplon

A. Weigh the Ocinaplon, Fast-flo lactose, Methocel K100M, Methocel K100LV, half of the Aerosil 200.

B. Blend in 10L V cone blender for ten minutes at 18 rpm.

C. Pass through a Fitzmill (hammers), 60 mesh screen, at high speed to produce particles having diameters less than 250 microns.

D. Granulate in a fluid bed system (Glatt GPCG-3), using PVP (7.5% w/w aq soln) as the binder, to a target composition of 5% binder/95% milled blend.

E. Blend granule in a V cone blender with remainder of the Aerosil, for 5 minutes, before adding Magnesium Stearate, and blending for a further 3 minutes.

F. Manufacture the SR tablets using a Piccola tablet Press, using 7 mm round, normal concave tooling to a target hardness of 200 N (±30%), and the required target weight (±7%).

G. Coat the resultant tablets with a cosmetic coating of Opadry Brown (12% w/w aq soln), on a Vector Coater, to a target weight gain of 3%.

### Example 3

### Preparation of a Bi-layer Tablet Having 30 mg SR/90 mg RR

The 90 mg IR composition of Example 1 and the 30 mg SR composition of Example 2 is used. Step A through E of examples 1 and 2 is utilized. The composition of Examples 1 and 2 are placed in a bi-layer tablet press and compressed in this press to form a single bi-layer tablet. After pressing, the tablets are coated in the manner of Step G. in Example 1.

### Example 4

### Procedure

Administration procedure utilizing a SR 30 mg tablet from Example 2 and a 90 mg RR tablet from Example 1.

The placebo for this study is prepared as below.

| | % BY WEIGHT BASED UPON |
|---|---|
| MATERIAL | WEIGHT OF COMPOSITION |
| Avicel | 96.6 |
| Mag Stearate | 0.5 |
| Opadry Brown | 2.9 |

Manufacturing Process

A. Weigh the Avicel, and the Magnesium Stearate. Blend together in a V cone blender at 18 rpm.

B. Manufacture the placebo tablets using a Piccola tablet Press, using 7 mm round, normal concave tooling or 9 mm round, normal concave tooling to the required hardness (+/−30%), and the required weight (+/−7%).

C. Coat the resultant tablets with a cosmetic coating of Opadry Brown (12% w/w aq soln), on a Vector Coater, to a target weight gain of 3%.

The actual study is run for 14 days. The total number of patients per treatment group are 60 patients. There are three treatment groups. The Ocinaplon (RR and SR combinations) and matching placebos are dosed as follows:

| | Total SR/RR Dose | | Total Daily |
|---|---|---|---|
| SR(mg/IR(mg) | (mg) | Regimen | Dose (mg) |
| 0/60 | 60 | Three times daily | 180 |
| 30/90 | 120 | Twice daily | 240 |
| Placebo | 0 | To match | 0 |

In order to maintain patient blinding, all patients are administered two tablets three times a day. The 30/90 Ocinaplon group receive a placebo as one of the three doses during the day; the other two doses which they receive contain the active ingredient.

### Example 5

Preparation of 85, 210, 195 and 195 mg SR Tablets

Tablets containing 85 mg, 110 mg, 195 mg and 210 mg were prepared from the ingredients listed in the table. In this table all % are in % by weight based upon the total weight of the composition.

| | A | B | C | D | E |
|---|---|---|---|---|---|
| Ocinaplon | 29.1% | 30.0% | 30.0% | 30.0% | 30.0% |
| Methocel | 9.3% | | | | 20.4% |
| K100M | | | | | |
| Methocel | 19.8% | 30.0% | 30.0% | 30.0% | 9.6% |
| K100LV | | | | | |
| Sodium Lauryl | — | — | 6.0% | 6.0% | 6.0% |
| Sulphate | | | | | |
| Lactose | 32.1% | 35.0% | 29.0% | 29.0% | 29.0% |
| Aerosil 200 | 1.0% | 1.0% | 1.0% | 1.0% | 1.0% |
| Kollidon 30 | 4.8% | 3.0% | 3.0% | 3.0% | 3.0% |
| Magnesium | 1.0% | 1.0% | 1.0% | 1.0% | 1.0% |
| Stearate | | | | | |
| Opadry Brown | 2.9% | | | | |
| Milligrams of | 210 mg | 85 mg | 85 mg | 110 mg | 195 mg |
| Ocinaplon | | | | | |
| per tablet | | | | | |
| Tablet | 18 × 8 mm. | 7 mm. | 7 mm. | 9 mm. | 18 × 8 mm. |
| Dimension | oval normal | round | round | round | oval |
| | concave | normal | normal | normal | normal |
| | | concave | concave | concave | concave |

The tablets were manufactured in a manner similar to Example 1, except that granulation was performed to achieve a theoretical composition of 3% binder and 97% milled blend for B-E. The tablets were not coated.

### Example 6

### Dissolution of Sustained Release Tablets Containing Ocinaplon

Dissolution Testing of the tablets of Example 2 and Example 5 (A), (B), (C), (D) and (E), was performed using USP 1 Apparatus, 20 mesh baskets, 100 rpm, 100 ml phosphate buffer pH 6.8±0.05 with 0.5% SLS, 37° C.±0.5° C.

The content of Ocinaplon in the sample was determined by HPLC, and is reported in the table below.

| | Example | Example | Example | Example | Example | Example |
|---|---|---|---|---|---|---|
| | 2 | 5 (A) | 5 (B) | 5 (C) | 5 (D) | 5 (E) |
| Time (Hrs) | Mean % Released | | | | | |
| 1 | 10.5 | 6.0 | 11.1 | 9.1 | 10.0 | 5.1 |
| 4 | 48.7 | 29.8 | 45.2 | 46.2 | 44.4 | 27.3 |
| 8 | 90.9 | 60.9 | 84.0 | 91.0 | 85.2 | 57.7 |
| 12 | 107.0 | 80.1 | 105.0 | 101.7 | 97.6 | 79.4 |
| 22 | — | 103.6 | 102.8 | 103.1 | 100.3 | 102.0 |

### Example 7

### Preparation of a 30 mg Rapid Release IR Tablet

Tablets containing 30 mg of Ocinaplon were prepared from the ingredients listed in the table below. All % are in % by weight based upon the total weight of the composition.

| Material | A |
|---|---|
| Ocinaplon | 9.4 |
| Fast Flow Lactose | 74.8 |
| Polyplasdone | 9.7 |
| Aerosil | 0.6 |
| Kollidon 30 | 1.6 |
| Mag Stearate | 1.0 |
| Opadry Brown | 2.9 |
| Milligrams of Ocinaplon per tablet | 30 |
| Tablet Dimension | 9 mm round |

### Example 8

### Preparation of a 60 mg and 25 mg RR Capsule

Tablets containing 60 mg of Ocinaplon (A) and 25 mg of the active ingredient (b) were prepared using the following table. In this table all % are % by weight based upon the total weight of the composition.

| Material | A | B |
|---|---|---|
| Ocinaplon | 26.25 | 26.25 |
| Avicel PH101 | 20.0 | 20.0 |
| Starch 1500 | 10.0 | 10.0 |
| Aerosil 200 | 0.25 | 0.25 |
| Pharmatose DCL 11 | 42.5 | 42.5 |
| Magnesium Stearate | 1.0 | 1.0 |

The excipients were sieved. All of the excipients except the Magnesium Stearate were blended for 15 minutes in a Pharmatech Blender equipped with a 10L V cone blender at 18 rpm. The Magnesium Stearate was added, and blended for a further 5 minutes. The blend was filled into capsules manually.

### Example 9

### Dissolution of Rapid Release Tablets and Capsules Containing Ocinaplon

| | | | | Example | Example |
|---|---|---|---|---|---|
| | Example 1 | Example 1 | Example 7 | 8A | 8B |
| | (60 mg IR | (90 mg IR | (30 mg IR | (60 mg IR | (25 mg IR |
| | Tablet) | Tablet) | Tablet) | Capsule) | Capsule |
| 0.5 hrs | 105.8% | 96.9% | 104.0% | 78.3% | 79.6% |

Dissolution testing of Example 1, Example 7 and Example 8(A) and (B) was performed using USP 1 Apparatus, 20 mesh blankets, 100 rpm, 0.01 N HCl.

What is claimed is:

1. A method for treating anxiety in a patient in need of said treatment comprising orally administering to said patient as an active ingredient, an anti-anxiety compound of the formula 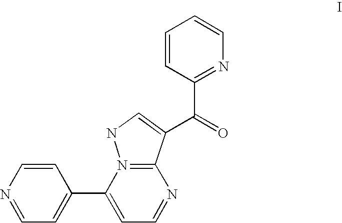
or a pharmaceutically acceptable salt thereof, at a daily dosage of from about 50 to 250 mg, with said daily oral dose having a first portion of the active ingredient in an rapid release form and the remaining portion of said active ingredient in a sustained release form, said proportion of said active ingredient in the rapid release form administered being from about 1 to about 4 times the weight of the portion administered in sustained release form.

2. The method of claim 1, wherein the daily dose is administered in from 1 to 3 administrations per day.

3. The method of claim 1, wherein said active ingredient is administered as tablets.

4. The method of claim 2, wherein each of said separate administrations, the sustained release portion is administered in combination with the rapid release portion.

5. The method of claim 4, wherein the daily dose of said anti-anxiety compound is from about 120 to 240 mg.

6. The method of claim 5, wherein in each of said administrations the proportion of said active ingredient in the rapid release portion form is about 2.5 to 3.5 times the weight of the portion in the slow release portion.

7. The method of claim 3, wherein in each of said administrations the slow release portion is administered together with the rapid release portions.

8. The method of claim 7, wherein in each of said administrations, the slow release portion and the rapid release portion are administered in a single tablet.

9. The method of claim 8, wherein said tablet contains the active ingredient rapid release form in an amount of about 3 times the weight of the active ingredient slow release form.

10. The method of claim 9, wherein the tablets administered contain about 10 mg of the active ingredient in its sustained release form and about 30 mg of the active ingredient in its rapid release form.

11. The method of claim 9 wherein the tablets administered contain about 30 mg of the active ingredient in its sustained release form and about 90 mg of the active ingredient in its rapid release form.

12. A pharmaceutical oral unit dosage form comprising two separate compartments each containing a composition comprised a pharmaceutically active ingredient selected from the group consisting of the compound of the formula 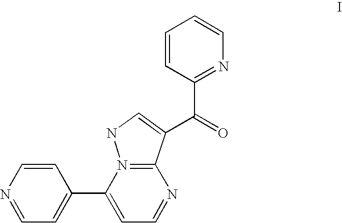
and a pharmaceutically acceptable salt thereof in a mixture with a pharmaceutically acceptable carrier, said active ingredient being present in the unit dosage form in an amount of from about 50 to about 250 mg, with the amount of said ingredient in the first compartment being from about 1 to about 4 times the weight of said active ingredient in the second compartment, the composition in the first compartment being adapted for rapid release of said active ingredient contained therein and the composition in the second compartment having incorporated therein a hydrophilic polymeric matrix which causes sustained release of the active ingredient in the second compartment.

13. The unit dosage form of claim 12, wherein the oral dosage form is a tablet.

14. The unit dosage form of claim 13, wherein the composition has a particle size diameter less than 250 microns.

15. The unit dosage form of claim 14, wherein the polymeric matrix is hydroxypropyl methyl cellulose.

16. The unit dosage form of claim 15, wherein the pharmaceutically acceptable carrier in each of said compartments is fast flow lactose.

17. The unit dosage form of claim 14, wherein the active ingredient is present in the unit dosage form in an amount of from about 80 to about 240 mg.

18. The unit dosage form of claim 17, wherein the active ingredient is present in the unit dosage form in an amount of from about 120 to about 240 mg.

19. The unit dosage form of claim 18, wherein the active ingredient is in the rapid release portion is in an amount of about 2.5 to 3.5 times the weight of the active ingredient in the sustained release portion.

20. The unit dosage form of claim 19, wherein the tablet contains about 30 mg of the active ingredient in the sustained release form and about 90 mg of the active ingredient in the rapid release form.

21. The unit dosage form of claim 20 wherein the tablet contains from about 10 mg of the active ingredient in sustained release form and about 30 mg of the active ingredient in rapid release form.
